(19) United States
(12) Patent Application Publication
(10) Pub. No.: US 2005/0256478 A1
(43) Pub. Date: Nov. 17, 2005

(54) Absorbent article having an outer layer with a hydrophilic region

(76) Inventor: Nathan A. Genke, Menasha, WI (US)

Correspondence Address: KIMBERLY-CLARK WORLDWIDE, INC., 401 NORTH LAKE STREET, NEENAH, WI, 54956, US

(21) Appl. No.: 10/836,491

(22) Filed: Apr. 29, 2004

(51) Int. Cl. A61F 13/15; A61F 13/20

(52) U.S. Cl. 604/385.01; 604/378; 604/385.23; 604/367

(57) ABSTRACT

An absorbent article defining an interior article surface and an exterior article surface opposite the interior surface. The article includes an absorbent body disposed toward the interior article surface, and an outer layer forming at least a portion of the exterior article surface. The article also includes a liquid impermeable layer located between the absorbent body and the outer layer; and at least one graphic disposed on the exterior article surface where the outer layer defines a hydrophilic region that is coextensive with at least a portion of the at least one graphic.

### FIELD OF THE INVENTION

This invention relates generally to absorbent articles such as training pants, diapers, feminine hygiene products, incontinence garments and the like. In particular, the present invention relates to absorbent articles that are configured to be worn in water (i.e. at the pool or the beach), and more particularly to such articles having an outer layer including a hydrophilic region.

### BACKGROUND OF THE INVENTION

Absorbent articles, particularly disposable absorbent articles such as training pants, diapers, incontinence products and the like are generally very effective at containing and absorbing body exudates. Nonetheless, conventional absorbent articles may not be completely suited for all types of activities and environments. For example, due to their considerable absorbent capabilities, conventional absorbent articles are not always completely satisfactory for use at a pool, at the beach, or other environment where the article may be submerged or otherwise have the exterior of the article exposed to a large amount of moisture.

Accordingly, articles that are specifically configured for use to be worn as swimwear are known in the art. For example, such articles may include an absorbent body that is configured not to significantly swell when submerged, and therefore are more readily worn and are more comfortable to maintain about the lower torso of the wearer. As such, these swimwear articles can, in certain instances, provide sufficient absorbent characteristics while performing an important waste containment function while the wearer is swimming or otherwise enjoying themselves. Nonetheless, such absorbent swimwear articles have not always been completely satisfactory.

That is, such articles may not provide the desired discretion to the wearer since such articles may not feature characteristics of swimwear articles such as multicolored graphics or a clothing-like texture and appearance. In particular, it is desired that such articles include festive graphics on the articles that correspond to beach or pool type usage. Further, it has been found that in some instances, where such articles include a cloth-like exterior and graphics, when the articles are submerged in water or other liquids, the cloth-like outer layer can obscure some or all of the graphics visible from the exterior of the article.

There is need, therefore, to provide an absorbent article that includes at least one graphic, is clothing-like in look and feel, and includes an outer layer that maintains the visibility of the graphic even after the article has been submerged in water or other liquids.

### SUMMARY OF THE INVENTION

In one aspect, the present invention is directed to an absorbent article that defines an interior article surface and an exterior article surface opposite the interior surface. The article includes an absorbent body disposed toward the interior article surface, an outer layer forming at least a portion of the exterior article surface, and a liquid impermeable layer located between the absorbent body and the outer layer. The article also includes at least one graphic disposed on the exterior article surface where the outer layer defines a hydrophilic region that is coextensive with at least a portion of the at least one graphic.

In another aspect, the present invention is directed to a disposable absorbent article that defines an interior article surface and an exterior article surface opposite the interior surface, a first waist region, a second waist region, and a crotch region connecting the first and second waist regions. The article includes an absorbent body disposed toward the interior article surface, an outer layer forming at least a portion of the exterior article surface, and a liquid impermeable layer located between the absorbent body and the outer layer. The article also includes at least one graphic disposed on the exterior article surface where the outer layer defines a hydrophilic region that is coextensive with at least a portion of the at least one graphic, and side panels extending from and connecting the first and second waist regions in a pant configuration to form a waist opening and a pair of leg openings.

In certain arrangements, the article of the present invention can be particularly suited for use as a swimwear article or the like. In such configurations, the absorbent body of the article can include less than 30 weight percent super absorbent material. Further, the absorbent body may optionally define a wet bulk thickness of less than 1 cm as determined by the Bulk Thickness test set forth herein.

The above-mentioned and other aspects of the present invention will become more apparent, and the invention itself will be better understood by reference to the drawings and the following description of the drawings.

### BRIEF DESCRIPTION OF THE DRAWINGS

FIG. 1 representatively illustrates a side view of a pair of swim pants shown joined on one side of the swim pants and open on the other side of the swim pants;

FIG. 2 representatively illustrates a plan view of a pair of swim pants similar to those illustrated in FIG. 1 in a opened, stretched and laid flat condition, and showing the surface of the swim pants that faces away from the wearer;

FIG. 3 representatively illustrates a plan view similar to FIG. 2, but showing the surface of the swim pants that faces the wearer when worn, and with portions cut away to show underlying features;

FIG. 4 representatively illustrates a partial section view of the swim pants;

FIG. 5 representatively illustrates an aspect of the swim pants of the present invention including a garment shell;

FIG. 6 representatively illustrates an inclined platform used in connection with the Hydrophilicity Test set forth herein.

Corresponding reference characters indicate corresponding parts throughout the drawings.

### DETAILED DESCRIPTION

“Attach” and its derivatives refers to the joining, adhering, bonding, connecting, or the like, of two elements. Two elements will be considered to be attached together when they are integral with one another, or attached directly to one another or indirectly to one another, such as when each is directly bonded to intermediate elements.

“Boxer shorts” or “Shorts” refers to a pant, trunks, briefs, and the like, that may be snug or loose fitting at the leg area.

The terms “comprises” and “comprising” as used herein have the same meaning as the terms “includes” and “including”. For example, the term “comprising” as employed in the claims specifies the presence of the stated features, elements, integers, steps, or components, but does not preclude the presence or addition of one or more other features, elements, integers, steps, components, or groups thereof.

“Connected” refers to the joining, adhering, bonding, attaching, or the like, of two elements. Two elements will be considered to be connected together when they are integral with one another or connected directly to one another or indirectly to one another, such as when each is directly connected to intermediate elements.

“Disposable” includes being disposed of after use and not intended to be washed and reused.

“Disposed,” “disposed on,” “disposed with,” “disposed toward,” “disposed near” and variations thereof are intended to mean that one element can be integral with another element, or that one element can be a separate structure attached to or placed with another element or placed near another element.

“Elastic,” “elasticized” and “elasticity” mean that property of a material by virtue of which it tends to recover its original size and shape after removal of a force causing a deformation.

“Hanging Legs” refers to the characteristic of a garment where the garment includes material that extends below the crotch of the garment and is intended to generally cover at least a portion of the leg of the wearer; the material may be loose fitting about the leg of the wearer or fit snugly about the leg of the wearer.

“Hydrophilic” describes the degree of wetting of a material by aqueous liquids in contact with the material. For example, the degree of wetting of the material may be quantified via the Hydrophilicity Test set forth herein. In particular and as used herein, a material is “hydrophilic” if it has a runoff value of less than 10 grams of runoff as determined by the Hydrophilicity Test set forth herein. It should be noted that the runoff value is determined by an average of at least 3 specimens of a sample in order to account for material variability.

“Integral” is used to refer to various portions of a single unitary element rather than separate structures bonded to or placed with or placed near one another.

“Inward” and “outward” refer to positions relative to the center of an absorbent garment, and particularly transversely and/or longitudinally closer to or away from the longitudinal and transverse center of the absorbent garment.

“Layer” when used in the singular can have the dual meaning of a single element or a plurality of elements.

“Liquid” means a substance and/or material that flows and can assume the interior shape of a container into which it is poured or placed.

“Liquid impermeable” when used to describe a layer or laminate that liquid such as urine will not pass through the layer or laminate under ordinary use conditions in a direction generally perpendicular to the plane of the layer or laminate at the point of liquid contact.

“Member” when used in the singular can have the dual meaning of a single element or a plurality of elements.

“Nonwoven” means a material which is formed without the aid of a textile weaving or knitting process.

“Operatively associated” refers to two or more components which act together.

“Pants” includes full length, short pants, and garments intended to be worn about the hips and lower torso of the wearer.

“Particles” refers to any geometric form such as, but not limited to, spherical grains, cylindrical fibers or strands, flat surfaces or roughened surfaces, sheets, ribbons, strings, strands, or the like.

“Releasably attached,” “releasably bonded,” “releasably engaged” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment.

“Superabsorbent” refers to a water-swellable, water-insoluble organic or inorganic material capable, under the most favorable conditions, of absorbing at least about fifteen times its weight and, more suitably, at least about thirty times its weight in an aqueous solution containing 0.9 weight percent sodium chloride. The superabsorbent materials can be natural, synthetic or modified natural polymers and materials, or a combination of such materials. In addition, the superabsorbent materials can be inorganic materials, such as silica gels, or organic compounds such as cross-linked polymers.

“Surface” includes any layer, film, woven, nonwoven, laminate, composite, or the like, whether pervious or impervious to air, gas, and/or liquids.

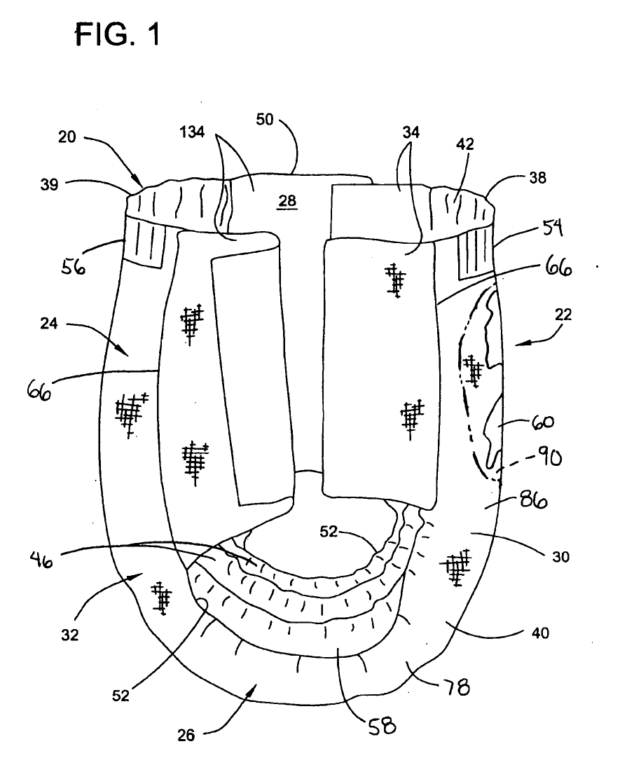

Referring now to the drawings and in particular to FIG. 1, a disposable swimwear article in the form of children's swim pants is indicated in its entirety by the reference numeral 20. The article may or may not be absorbent, which generally refers to absorbent articles that may be placed against or in proximity to the body of the wearer to absorb and/or retain various liquid waste discharged from the body. It is understood that the present invention is suitable for use with various absorbent articles such as training pants, diapers, feminine hygiene products, incontinence products, medical articles such as medical garments, surgical pads and bandages, other personal care or health care garments, apparel for institutional, industrial or consumer use, and the like without departing from the scope of the present invention. In addition, it is understood that the present invention may be used with various other swimwear articles such as swim trunks, swimsuits including a skirt and/or bodice, adult swimwear articles, and the like without departing from the scope of the present invention.

By way of illustration only, articles suitable for use with the present invention and various materials and methods for constructing such garments are disclosed in U.S. Pat. No. 6,497,694 issued Dec. 24, 2002 to Rosch et al.; PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al.; and U.S. Pat. No. 6,645,190 issued Nov. 11, 2003 to Olson et al., the disclosures of which are hereby incorporated by reference to the extent that they are consistent (i.e., not in conflict) herewith. In addition, it should be understood that the various components of the different aspects of the swim pants 20 can be attached together using adhesives, thermal bonds, mechanical bonds, or other means known to those skilled in the art.

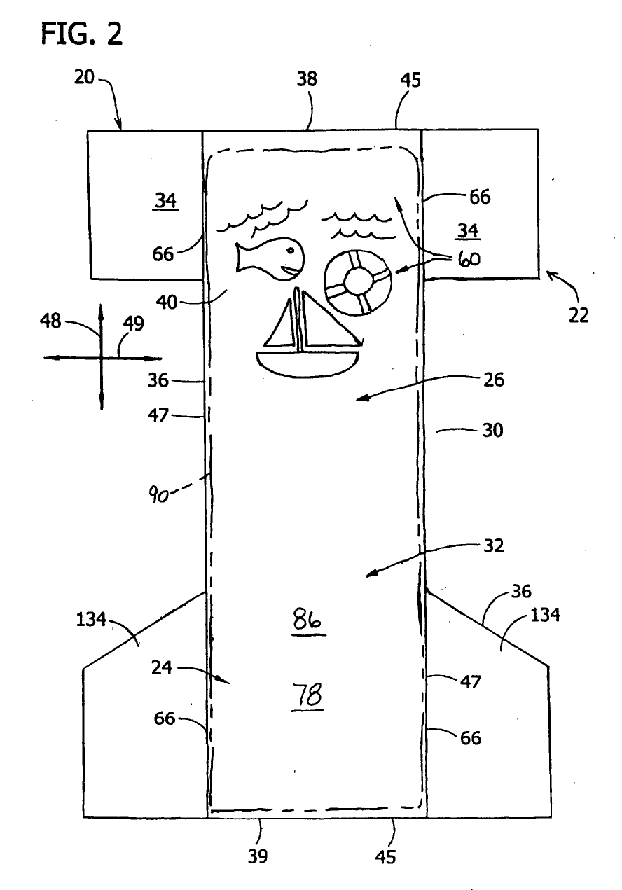

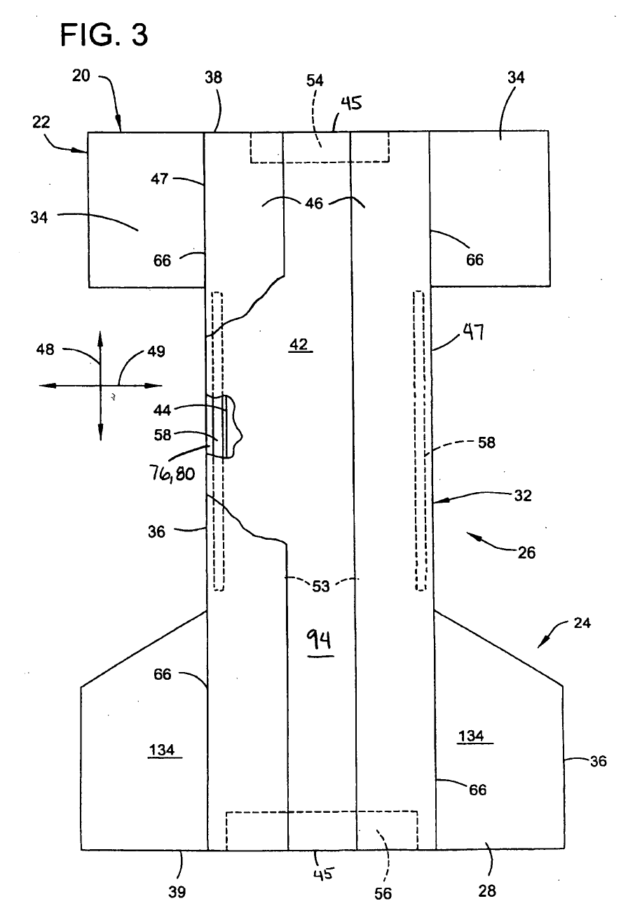

One aspect of the swim pants 20 is illustrated in FIG. 1 in a partially fastened condition and includes a first waist region and a second waist region, i.e., the front waist region 22, and the back waist region 24. The swim pants 20 also include a crotch region 26 interconnecting the front and back waist regions 22 and 24, an interior article surface 28 configured to be positioned toward the wearer in use, and an exterior article surface 30 opposite the interior article surface 28. With additional reference to FIGS. 2 and 3, the swim pants 20 also can have a pair of laterally opposite side edges 36 and a pair of longitudinally opposite waist edges, respectively designated front waist edge 38 and back waist edge 39. The swim pants 20 also include at least one graphic 60 and an outer layer 78 having at least one hydrophilic region 90.

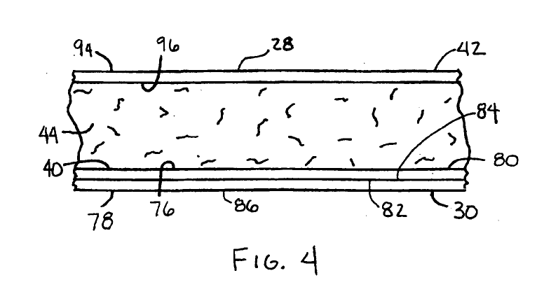

The illustrated pants 20 can include a central absorbent assembly, generally indicated at 32, which when laid flat can be rectangular or any other desired shape. The swim pants 20 may also include a pair of laterally opposite front side panels 34 extending outward from the absorbent assembly 32 at the front waist region 22 and a pair of laterally opposite back side panels 134 extending outward from the absorbent assembly at the back waist region 24. The absorbent assembly 32 and side panels 34, 134 may comprise two or more separate elements, as representatively illustrated in FIGS. 1-3, or may be integrally formed. The central absorbent assembly 32 may include a liquid impermeable layer 76, a bodyside liner 42 (FIGS. 1, 3 and 4) attached to the liquid impermeable layer 76 in a superposed relation, and an absorbent body 44 (FIGS. 3 and 4) disposed between the liquid impermeable layer 76 and the bodyside liner 42. Accordingly, the liquid impermeable layer 76 may be located between the absorbent body 44 and the outer layer 78.

The absorbent assembly 32 also has opposite ends 45 that can form portions of the front and back waist edges 38 and 39, and opposite side edges 47 that can form portions of the side edges 36 of the swim pants 20 (FIGS. 2 and 3). Integrally formed side panels 34, 134 and absorbent assembly 32 would include at least some common materials, such as the bodyside liner 42, liquid impermeable layer 76, other materials and/or combinations thereof, and could define a one-piece elastic, stretchable, or nonstretchable pants. For further reference, arrows 48 and 49 depict the orientation of the longitudinal axis and the transverse or lateral axis, respectively, of the swim pants 20 (FIGS. 2 and 3).

With the swim pants 20 in the closed or fastened position as partially illustrated in FIG. 1, the front and back side panels 34, 134 are connected together to define a three-dimensional pants configuration having a waist opening 50 and a pair of leg openings 52. The front waist region 22 comprises the portion of the swim pants 20 which, when worn, is positioned on the front of the wearer while the back waist region 24 comprises the portion of the swim pants which is positioned on the back of the wearer. The crotch region 26 of the swim pants 20 comprises the portion of the swim pants 20 which is positioned between the legs of the wearer and covers the lower torso of the wearer. The front and back side panels 34 and 134 comprise the portions of the swim pants 20 which, when worn, are positioned on the hips of the wearer. The waist edges 38 and 39 of the swim pants 20 are configured to encircle the waist of the wearer and together define the waist opening 50 (FIG. 1). Portions of the side edges 36 in the crotch region 26 generally define the leg openings 52.

The swim pants 20 can be configured to contain and/or absorb exudates discharged from the wearer. For example, the swim pants 20 may include containment flaps 46 that are configured to provide a barrier to the transverse flow of body exudates (FIGS. 1 and 3). The containment flaps 46 define a partially unattached edge which assumes an upright configuration in at least the crotch region 26 of the swim pants 20 to form a seal against the wearer's body. Suitable constructions and arrangements for the containment flaps 46 are generally well known to those skilled in the art and are described in U.S. Pat. No. 4,704,116 issued Nov. 3, 1987 to Enloe, the disclosure of which is hereby incorporated by reference to the extent that it is consistent (i.e., not in conflict) herewith.

To further enhance containment and/or absorption of body exudates, the swim pants 20 may also suitably include a front waist elastic member 54, a rear waist elastic member 56, and leg elastic members 58, as are known to those skilled in the art (FIGS. 1 and 3). The waist elastic members 54 and 56 can be operatively joined to the liquid impermeable layer 76 and/or the bodyside liner 42 along the opposite waist edges 38 and 39, and can extend over part or all of the waist edges 38 and 39. The leg elastic members 58 can be operatively joined to the liquid impermeable layer 76 and/or the bodyside liner 42 along the opposite side edges 36 and positioned in the crotch region 26 of the swim pants 20. The leg elastic members 58 can be longitudinally aligned along each side edge 47 of the absorbent assembly 32.

The waist elastic members 54 and 56 and the leg elastic members 58 can be formed of any suitable elastic material. As is well known to those skilled in the art, suitable elastic materials include sheets, strands or ribbons of natural rubber, synthetic rubber, or thermoplastic elastomeric polymers. In one particular aspect, for example, the leg elastic members 58 comprise a plurality of dry-spun coalesced multifilament spandex elastomeric threads sold under the trade name LYCRA® and available from E. I. Du Pont de Nemours and Company, Wilmington, Del., U.S.A.

The liquid impermeable layer 76 can be both liquid and vapor impermeable, or can be liquid impermeable and vapor permeable. The liquid impermeable layer 76 is desirably manufactured from a thin plastic film, although other flexible liquid impermeable materials can also be used. The liquid impermeable layer 76 prevents waste material from wetting articles, such as bed sheets and clothing, as well as the wearer and caregiver. Examples of a suitable liquid impermeable film for use as the liquid impermeable layer 76 are a 0.025 millimeter (1.0 mil) polyethylene film commercially available from Edison Plastics Company of South Plainfield, N.J., or a 0.019 millimeter (0.75 mil) EMB-921 film material commercially available from Tredegar Corporation, Richmond Va. A suitable microporous, or “breathable” film is a PMP-1 film material commercially available from Mitsui Toatsu Chemicals, Inc., Tokyo, Japan, or an XKO-8044 polyolefin film commercially available from 3M Company, Minneapolis, Minn.

The liquid permeable bodyside liner 42 is representatively illustrated as being in superposed relationship with the liquid impermeable layer 76, and with the liquid impermeable layer 76 and the bodyside liner 42 sandwiching the absorbent body 44. The bodyside liner 42 may, but need not have the same dimensions as the liquid impermeable layer 76. The bodyside liner 42 may be disposed toward the interior article surface 28; for example, at least a portion of the bodyside liner 42 may provide the interior article surface 28. In particular, the bodyside liner 42 may define an interior liner surface 94 and an exterior liner surface 96 opposite the interior liner surface; accordingly, the interior liner surface 94 may provide at least a portion of the interior article surface 28.

The bodyside liner 42 is desirably compliant, soft feeling, and non-irritating to the child's skin. Further, the bodyside liner 42 can be less hydrophilic than the absorbent body 44, to present a relatively dry surface to the wearer and permit liquid to readily penetrate through its thickness. Alternatively, the bodyside liner 42 can be more hydrophilic or can have essentially the same affinity for moisture as the absorbent body 44 to present a relatively wet surface to the wearer to increase the sensation of being wet. This wet sensation can be useful as a training aid.

Various woven and nonwoven fabrics can be used for the bodyside liner 42. For example, the bodyside liner can be composed of a meltblown or spunbonded web of polyolefin fibers. The bodyside liner can also be a bonded-carded web composed of natural and/or synthetic fibers. The bodyside liner can be composed of a substantially hydrophobic material, and the hydrophobic material can, optionally, be treated with a surfactant or otherwise processed to impart a desired level of wettability and hydrophilicity. For example, the material can be surface treated with about 0.38 weight percent of a surfactant mixture comprising Ahcovel N-62 from Hodgson Textile Chemicals of Mount Holly, N.C., U.S.A. and Glucopan 220UP from Henkel Corporation of Ambler, Pa. in an active ratio of 3:1. A suitable liquid permeable bodyside liner 42 is a nonwoven bicomponent web having a basis weight of about 27 gsm. The nonwoven bicomponent can be a spunbond bicomponent web, or a bonded carded bicomponent web. Suitable bicomponent fibers include a polyethylene/polypropylene bicomponent fiber available from CHISSO Corporation, Osaka, Japan.

As discussed above, the absorbent body 44 (FIGS. 3 and 4) may be located between the liquid impermeable layer 76 and the bodyside liner 42. In particular, the absorbent body 44 may be disposed toward the interior article surface 28, particularly with reference to the liquid impermeable layer 76. The absorbent body 44 can be any structure which is generally compressible, conformable, non-irritating to the child's skin, and capable of absorbing and retaining liquids and certain body wastes, and may be manufactured in a wide variety of sizes and shapes, and from a wide variety of liquid absorbent materials commonly used in the art. For example, the absorbent may be a mix of pulp and polymer co-formed to give it durability when wet (“coform”). The coform material may comprise an airlaid blend of cellulosic wood fibers and meltblown polyolefin fibers, such as polyethylene or polypropylene fibers, or may comprise an air-formed batt of cellulosic fibers (i.e., wood pulp fluff). Optionally, the absorbent core 60 may be treated with a surfactant to aid in liquid acquisition when in a dry environment. Coform materials and methods of making coform materials are described in U.S. Pat. No. 4,100,324 issued to Anderson et al., and in U.S. Pat. No. 5,508,102, issued to Georger et al., the disclosures of which are hereby incorporated by reference to the extent that they are consistent (i.e., not in conflict) herewith.

Alternatively, the absorbent body 44 includes a matrix of hydrophilic fibers, such as a web of cellulosic fluff, mixed with particles of superabsorbent material. For example, a suitable cellulosic fluff may be derived from CR1654 wood pulp, available from U.S. Alliance, Childersburg, Ala., U.S.A. The superabsorbent materials can be suitably selected from natural, synthetic, and modified natural polymers and materials. Suitable superabsorbent materials are available from various commercial vendors, such as BASF Aktiengesellschaft located in Ludwigshafen, Germany, and Stockhausen GmbH & Co. KG, Krefeld, Germany. The absorbent body 44 may, in certain aspects, contain from 0 to 95 weight percent superabsorbent.

In particular aspects, the absorbent body 44 may suitably contain little or no superabsorbent material. As such, when the wearer of the swim pants 20 is in an aquatic environment (i.e. the pool, the beach, or the like), the absorbent body 44 will not significantly swell when submerged, and the swim pants 20 can be more readily and comfortably maintained about the lower torso of the wearer. In particular, the absorbent body 44 of the present invention may include less than 25 weight percent superabsorbent material. In another aspect, the absorbent body of the present invention may include less than 10 weight percent superabsorbent material. In yet another aspect, the absorbent body of the present invention may include less than 5 weight percent superabsorbent material. Alternatively, the absorbent body of the present invention may include between 0 and 25 weight percent superabsorbent material. In another alternative, the absorbent body of the present invention may include between 0 and 10 weight percent superabsorbent material. In yet another alternative, the absorbent body of the present invention may include between 0 and 5 weight percent superabsorbent material.

Similarly, in configurations where the absorbent body is arranged to minimize swelling upon being submerged or otherwise being exposed to large amounts of liquid, the absorbent body may define a particular bulk thickness when wet and when dry. For example, the absorbent body 44 may define a bulk thickness of less than 3.0 cm when dry. Alternatively, the absorbent body 44 may define a bulk thickness of less than 2.5 cm when dry. In still yet another alternative, the absorbent body 44 may define a bulk thickness of less than 1.25 cm when dry. Further, the absorbent body 44 may define a bulk thickness of less than 5.0 cm when wet. In particular, the absorbent body 44 may define a bulk thickness of less than 3.0 cm when wet, and still more particularly the absorbent body 44 may define a bulk thickness of less than 1.0 cm when wet.

The absorbent assembly 32 can also incorporate other materials or components designed primarily to receive, temporarily store, and/or transport liquid along with the absorbent body 44, thereby maximizing the absorbent capacity of the absorbent assembly 32. One suitable additional component is commonly referred to as a surge layer and comprises a material having a basis weight of about 50 to about 120 grams per square meter, and more particularly comprises a through-air-bonded-carded web of a homogenous blend of 60 percent 3 denier type T-256 bicomponent fiber comprising a polyester core/polyethylene sheath and 40 percent 6 denier type T-295 polyester fiber, both commercially available from Kosa Corporation of Salisbury, N.C., U.S.A.

As noted previously, the swim pants 20 may have front and back side panels 34 and 134 disposed on each side of the absorbent assembly 32. The side panels 34, 134 can be permanently attached along seams 66 to the central absorbent assembly 32 in the respective front and back waist regions 22 and 24. More particularly, as representatively illustrated in FIGS. 1-3, the front side panels 34 can be permanently attached to and extend transversely outward beyond the side edges 47 of the absorbent assembly 32 in the front waist region 22, and the back side panels 134 can be permanently attached to and extend transversely outward beyond the side edges of the absorbent assembly in the back waist region 24. The side panels 34 and 134 may be attached to the absorbent assembly 32 using attachment means known to those skilled in the art such as adhesive, thermal or ultrasonic bonding. Alternatively, the side panels 34 and 134 can be formed as an integral portion of a component of the absorbent assembly 32. For example, the side panels can comprise a generally wider portion of the liquid impermeable layer 76, the bodyside liner 42, and/or another component of the absorbent assembly 32. The front and back side panels 34 and 134 can be permanently attached together or be releasably connected with one another such as by a fastening system (not shown). A suitable fastening system is described in U.S. Pat. No. 6,645,190 to Olson, et al., previously incorporated herein.

The side panels 34, 134 suitably, although not necessarily, comprise an elastic material capable of stretching in a direction generally parallel to the transverse axis 49 of the swim pants 20. Suitable elastic materials, as well as one process of incorporating elastic side panels into articles, are described in the following U.S. Patents: U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; the disclosures of which are hereby incorporated by reference to the extent that they are consistent (i.e., not in conflict) herewith. In particular aspects, the elastic material may comprise a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; the disclosures of which are hereby incorporated by reference to the extent that they are consistent (i.e., not in conflict) herewith.

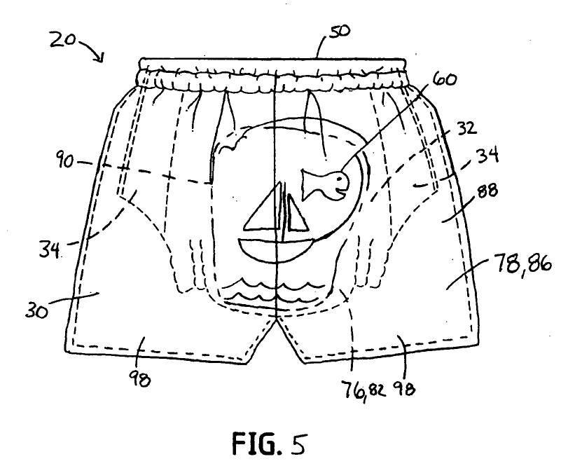

The swim pants 20 further include at least one graphic 60 disposed on the exterior article surface 30 of the pants 20. For example, the pants 20 may include a single graphic 60 disposed on the exterior article surface 30. Alternatively, as representatively illustrated in FIGS. 2 and 5, the pants 20 may include a plurality of graphics 60 disposed on the exterior article surface 30. Accordingly, by being disposed on the exterior article surface 30 of the pants 20, the graphics 60 are generally visible from the exterior article surface 30.

The graphics 60 can be formed on or applied to the swim pants 20 in a variety of ways such that the graphics 60 are disposed on the exterior article surface 30. For example, the graphics 60 may be applied directly or indirectly to any surface of the liquid impermeable layer 76 such as the interior liquid impermeable layer surface 80 or the exterior liquid impermeable layer surface 82. Alternatively the graphics 60 may be formed or applied between the liquid impermeable layer 76 and the absorbent body 44, formed or applied to a substrate that is placed with or near the liquid impermeable layer 76, formed or applied within the liquid impermeable layer 76, or other variations or combinations thereof.

In other aspects, the graphics 60 can be applied directly or indirectly to the outer layer 78. That is, the graphics may be applied directly or indirectly to the interior outer layer surface 84, the exterior outer layer surface 86, formed or applied to a substrate that is placed with or near the outer layer 78, formed or applied within the outer layer 78 or variations or combinations thereof.

Various placements of the graphics 60 may be better understood with reference to the partial section view of pants 20 that is shown in FIG. 4. FIG. 4 illustrates a partial section view of a swim pants 20 having an absorbent body 44 sandwiched between a liquid impermeable layer 76 and a bodyside liner 42. The swim pants 20 also include an outer layer 78. The liquid impermeable layer 76 has an interior liquid impermeable layer surface 80 and an opposite exterior liquid impermeable layer surface 82. In addition, the outer layer 78 has an interior outer layer surface 84 and an exterior outer layer surface 86, which also may provide at least a portion of the exterior article surface 30. The graphics 60 can be disposed on the liquid impermeable layer 76, which includes either surface 80 or 82 of the layer, on the surface of the absorbent body 44 that faces the liquid impermeable layer 76, or between the absorbent body 44 and the liquid impermeable layer 76. The graphics 60 may alternatively be disposed on the outer layer 78, which includes either surface 84 or 86 of the layer.

The graphics 60 of the present invention may include permanent graphics, active graphics, or combinations of active and permanent graphics. As used herein, the term “active graphic” refers to an appearing graphic, a fading graphic, or a combination of appearing and fading graphics. In contrast to active graphics, the term “permanent graphic” is used herein to refer to a graphic that does not substantially change its degree of visibility when the absorbent article is insulted with urine in simulated use conditions. The term “appearing graphic” is used herein to refer to a graphic that becomes visible or becomes significantly more visible when exposed to urine, or that becomes visible or becomes significantly more visible with the passage of time when exposed to the environment but not exposed to urine. Conversely, the term “fading graphic” is used herein to refer to a graphic that becomes invisible or significantly less visible when exposed to urine, or that becomes invisible or significantly less visible with the passage of time when exposed to the environment but not exposed to urine. Graphics suitable for use with the present invention are described in U.S. Pat. No. 6,297,424 issued Oct. 2, 2001 to Olson, et al. and U.S. Pat. No. 6,307,119 issued Oct. 23, 2001 to Cammarota et al., the disclosures of which are hereby incorporated by reference to the extent that they are consistent (i.e., not in conflict) herewith.

The graphics 60 may include, but are not limited to, scenes, characters, animals, objects, alphanumerics such as numbers, letters, words, phrases and the like, highlighting or emphasizing leg and waist openings 52, 50 in order to make product shaping more evident or visible to the user; highlighting or emphasizing areas of the product to simulate functional components such as elastic leg bands, elastic waistbands, simulated “fly openings” for boys, ruffles for girls; highlighting areas of the product to change the appearance of the size of the product; registering wetness indicators, temperature indicators, and the like in the product; registering a back label, or a front label, in the product; and registering written instructions at a desired location in the product. In particular aspects, the graphic 60 may also be gender specific; that is, the graphic 60 may, be a graphic that may be generally considered to be of interest to boys or to girls.

The graphics 60 may also include multicolored graphics as are suitable for swimwear articles. Alternatively, the graphics 60 may include generally monochrome graphics, such as monochrome silhouettes of objects and figures. Desirably, the graphics 60 are multicolored such that the swim pants 20 more closely resemble traditional swim trunks or bathing suits. As will be described below, the swim pants 20 of present invention can be particularly suited for having multicolored graphics 60.

As mentioned above, the graphics 90 may be disposed on the pants 20 using a variety of methods. For example, the graphics 60 and 90 may suitably be disposed on the pants 20 by being imprinted thereon using a flexographic printing process. Flexographic printing is a conventional printing technique which uses flexible, raised rubber or photopolymer plates to carry an inked image to a substrate, such as the bodyside liner 42, the liquid impermeable layer 76 or the outer layer 78 of the pants 20. As an example, flexographic printing apparatus are shown and/or described in U.S. Pat. No. 5,458,590 (Schleinz et al.); U.S. Pat. No. 5,566,616 (Schleinz et al.); U.S. 2003/0019374A1 (Harte); and U.S. Pat. No. 4,896,600 (Rogge et al.).

As mentioned above, the swim pants 20 of the various aspects of the present invention further includes an outer layer 78. The outer layer 78 of the present invention is generally disposed toward the exterior article surface 30 and may suitably be generally cloth-like in appearance and texture. For example, as representatively illustrated in FIGS. 1-5, the outer layer 78 may form at least a portion of the exterior article surface 30.

The outer layer 78 can be any suitable material and desirably one that provides a generally cloth-like appearance and texture, as mentioned above. The outer layer 78 may be a nonwoven material, or may alternatively be provided by natural fibers, or combinations thereof. One example of such a material is a 20 gsm (grams per square meter) spunbond polypropylene nonwoven web. The outer layer 78 can also be made of those materials of which liquid permeable bodyside liner 42 is made. Alternatively, the outer layer 78 may be provided by a different material than the bodyside liner 42.

In addition, the outer layer 78 may be configured to include at least one hydrophilic region 90 (FIGS. 1, 2, and 5). Alternatively, the outer layer 78 may include a plurality of hydrophilic regions 90, or in yet another alternative, substantially the entire outer layer may be configured to be hydrophilic and provide the hydrophilic region 90 (FIG. 2).

The hydrophilic region 90 of the outer layer 78 can be coextensive with at least a portion of the graphic 60. More suitably, and as representatively illustrated in FIGS. 1, 2 and 5, the hydrophilic region 90 is entirely coextensive with the graphic 60. Accordingly, the hydrophilic region 90 may be limited to a portion of the outer layer 78 and registered with the graphic 60, or alternatively, the hydrophilic region may include substantially the entire outer layer 78 for ease of manufacture and processing. Thus, the hydrophilic region 90 can be located as necessary along the length and width of outer layer 78 to maintain the desired visibility of the graphic 60 when the swim pants 20 have been submerged. That is, when the exterior article surface 30 of the swim pants 20 is submerged or otherwise is exposed to liquid and becomes wet, the graphic is more readily visible from the exterior article surface 30 than if the outer layer 78 did not include the hydrophilic region 90. Without being bound to any particular theory, it is believed that the hydrophilic region 90 of the outer layer 78 makes the graphic 60 more readily viewable when the exterior article surface 30 is wet because the liquid does not sit upon the top of the outer layer 78 and obstruct the graphic from the viewer. This is particularly advantageous where it is desired to have swim pants 20 with multicolored and/or sophisticated graphics including popular characters or figures. When the swim pants 20 include such graphics 60, and also include an outer layer with a hydrophilic region 90, these graphics remain visible when the outer surface 30 of the swim pants 20 are wet. Without an outer layer 78 that includes a hydrophilic region 90, the swim pants 20 may have to resort to monochrome or black silhouette graphics such that the graphics are more readily discernable when the outer surface 30 of the swim pants 20 are wet.

Accordingly, the hydrophilic region 90 of the outer layer 78 may define a runoff value of less than 10 grams of runoff as determined by the Hydrophilicity Test set forth herein. Alternatively, the hydrophilic region 90 defines a runoff value of less than 6 grams of runoff; in another alternative, the hydrophilic region 90 defines a runoff value of less than 5 grams of runoff; and in still yet another alternative, the hydrophilic region 90 defines a runoff value of less than 4 grams of runoff as defined by the Hydrophilicity Test set forth herein.

The hydrophilic region 90 may be provided on the outer layer 78 in a variety of ways as is known in the art. For instance, in one aspect, certain portions of the outer layer 78 may be configured to provide the desired hydrophilicity (that is, the desired runoff value), or alternatively, substantially the entire outer layer 78 may be configured to provide the desired hydrophilicity. In another aspect, the hydrophilic region 90 may include a nonwoven material or natural fibers or combinations thereof, which provide the desired hydrophilic characteristics. For example, the outer layer 78 may be a nonwoven material arising from a melt blend of polypropylene and a hydrophilic additive to provide a layer with the desired hydrophilicity. In particular, the outer layer 78 may be may a nonwoven material provided by a polypropylene and polyethylene glycol blend, with the polypropylene to polyethylene glycol ratio being at 95:5 (by weight).

Alternatively, the hydrophilic region 90 may include a surfactant applied to the outer layer 78 to achieve the desired hydrophilic characteristics. For example, the outer layer 78 can be surface treated with about 0.3 to about 0.45 weight percent of a surfactant mixture comprising Ahcovel N-62 from Hodgson Textile Chemicals of Mount Holly, N.C., U.S.A. and Glucopan 220UP from Henkel Corporation of Ambler, Pa. in an active ratio of 3:1. The surfactant can be applied by any conventional means, such as spraying, printing, brush coating or the like.

As representatively illustrated in FIGS. 1-4, the outer layer 78 may be provided in connection with the liquid impermeable layer 76 to provide a laminate outercover 40. Therefore, in such a configuration, at least a portion of the liquid impermeable layer 76 may be attached to the outer layer 78; for example, the layers 76 and 78 may suitably be substantially attached together, such as by a laminate adhesive (not shown). Suitable laminate adhesives, which can be applied continuously or intermittently as beads, a spray, parallel swirls, or the like, can be obtained from Findley Adhesives, Inc., of Wauwatosa, Wis., or from National Starch and Chemical Company, Bridgewater, N.J. Alternatively, other methods for joining the layers 76 and 78 may be used, such as thermal or ultrasonic bonding, pressure bonding and the like or combinations thereof.

In another aspect, as representatively illustrated in FIG. 5, the swim pants 20 may include a garment shell 88. As such, the outer layer 78 may be provided by at least a portion of the garment shell 88. Alternatively, the outer layer 78 may be applied to the exterior of the garment shell 88. Garment shell configurations suitable for use with the present invention, and methods of manufacturing them, are described in U.S. Pat. No. 6,192,521 issued Feb. 27, 2001 to Alberts et al. the disclosure of which is hereby incorporated by reference to the extent that it is consistent (i.e., not in conflict) herewith. For example, the garment shell 88 may be provided in a boxer short configuration and include a waist opening 50 and a pair of hanging legs 98. In another alternative, the garment shell 88 may be in the form of a skirt-like wrap that may encircle the absorbent chassis 32.

Materials suitable for the garment shell 88 include the materials described herein as appropriate for use as the liquid impermeable layer 76 and the outer layer 78. Alternatively, the garment shell 88 may be provided by a variety of materials as are known in the art. For instance, the garment shell may be provided by a nonwoven web, plastic film layers, and the like or combinations thereof. In particular, the garment shell may include a laminate of a nonwoven layer such as spunbond polypropylene of 20 to 70 grams per square meter and a polyethylene film layer of 20 to 70 gsm.

### Hydrophilicity Test

With reference to FIG. 6, an inclined platform 110 can be used to conduct the hydrophilicity testing. The platform 10 includes a base 12 and an inclined surface 14. The inclined surface 14 has a width of 35 centimeters and a length along its transverse centerline of 59 centimeters. The inclined surface shown is inclined at an angle of 30 degrees. Located at a bottom edge 16 of inclined plane 14 are V-shaped barriers 18. Barriers 18 serve to funnel liquid running down inclined surface 14 into a hole 20 located in the center of barriers 18. Scribed on the surface of inclined surface 14 is a line, 25 centimeters from the top (horizontal) edge of inclined surface 14. The scribed line is parallel to the top edge of inclined surface 14. A collection vessel (not shown) is placed underneath the hole 20 to collect any liquid that is not absorbed by the specimen being tested. A layer of water impermeable material (not shown) is used in the bottom section of inclined surface 14 and overlaid atop barriers 18 to isolate any test liquid (runoff) that does not pass through the material specimen being tested. A second collection vessel (not shown) is used to collect this runoff. Suspended above inclined surface 14 is a dispensing funnel 22. Dispensing funnel 22 is adapted to hold 50 milliliters of a liquid, which liquid can be released through valve 24 onto inclined surface 14. The height of valve 24 above inclined surface 14 is adjustable to allow for a clearance of 20 millimeters between the bottom edge of valve 24 and a specimen to be tested when in position on inclined surface 14.

Specimen Preparation:

Each test specimen is composed of a material specimen, which is a piece of outer layer 78 of the sample being tested, overlaid on an absorbent layer (see below). The outer layer 78 to be tested should be free of attachment to any other layer. Care should be taken at all times prior to testing to prevent exposure of the outer layer 78 to moisture or contamination, and the layer should not be folded, torn, crumpled or creased in any region to be included in a material specimen. The specimen should be subjected to minimal handling at all times prior to testing. Where it is not possible to obtain the layer prior to attachment to other materials (such as when an intact product is to be tested), the following procedure should be used to separate the outer layer from other material layers: Gentle heating may be used to soften any adhesive. The outer layer 78 should then be gently separated from adjoining layers. Care should be taken to avoid disturbing the structure of the material specimen, and to minimize handling of the specimen. The specimen should not be torn. The specimen should not be exposed to solvents in order to soften adhesives. The interior outer layer surface 84 should be indicated on the specimen, using a small mark in one corner of the specimen. The specimen dimensions are 14 centimeters wide by at least 27 centimeters long. It may be possible to obtain more than one specimen from a single product; however, the area of the outer layer 78 that overlies the graphic 60 should be selected for testing. The area over the graphic should be centered in the material specimen dimensions, if possible.

Test Procedure:

A generally rectangular test specimen 14 centimeters wide and 27 centimeters long is prepared in the following manner. An absorbent layer is prepared to be 14 centimeters wide and at least 25 centimeters long. A suitable absorbent material may be smooth toweling such as VIVA® Paper Towels manufactured by Kimberly-Clark Corporation of Neenah, Wis. At least one towel may be individually torn from the roll along perforations, and folded once along its centerline perpendicular to the perforation lines. The number of towels may be selected to provide best absorbency of liquid that passes through the test material, or to provide a desired thickness of the test specimen (desired distance from bottom edge of valve 24). The smoothest side of each towel should face outward. The folded towel(s) may be stacked with their edges aligned to provide an absorbent pad. If the towels buckle or curl, or otherwise do not lie flat due to the fold, each towel can be slit along its fold.

A material specimen is smoothly overlaid on top of the absorbent layer, with edges aligned with the absorbent layer on both sides and at one narrow edge. The material specimen should extend at least about 0.5 cm beyond the other narrow end of the absorbent layer. Care should be taken to place the interior outer layer surface 84 (FIG. 4) facing down, against the absorbent.

The test specimen is then mounted on inclined surface 14 using tape at each upper corner, or by clamping the specimen to the upper edge of inclined surface 14. The test specimen is generally centered widthwise on inclined surface 14 and the funnel 22 is directed at a point approximately 120 millimeters from the bottom (lowest edge) of the test sample and transversely centered on the specimen. The narrow edge of the test specimen on which the material specimen hangs beyond the absorbent by about 0.5 cm should be placed at the lower end of the incline. The lower edge of the absorbent layer should be aligned with the scribe line; the lower edge of the material specimen will hang below the scribe line. The valve 24 is located approximately 20 millimeters above the top surface of the specimen.

A piece of flexible, water impermeable material at least as wide as the material specimen and long enough to extend past the V-shaped barriers 18 of the test apparatus is then applied to the lower edge of the material specimen that extends beyond the absorbent. A piece of film-type tape such as HIGHLAND® Invisible Tape, available from 3M Corporation of St. Paul, Minn., should be applied across the upper edge of the water impermeable material and the lower edge of the material specimen so as to join them together. The tape is applied perpendicular to the long dimension of the material sample, and should be of sufficient length to extend beyond the lateral edges of the material sample and absorbent. The upper long edge of the tape should be aligned with the underlying edge of the absorbent material beneath the material specimen (i.e., aligned with the scribe line on inclined surface 14). The segments of tape beyond the lateral edges of the test specimen should be carefully applied to the face of inclined surface 14, in such a way as to draw the material specimen flat (without stretching), and to avoid ripples or uneven spots in the specimen. The tape should additionally provide a smooth junction between material specimen and water impermeable material, so that any runoff can flow unimpeded from the face of the specimen onto the water impermeable material, if needed. Barriers can be raised at the lateral edges of the water impermeable material if needed, to aid in channeling all runoff into the appropriate collection vessel.

The recommended test liquid is distilled water. Fifty milliliters (50+/−0.5 mL) of distilled water is placed in funnel 22. The water should be equilibrated to reasonable ambient conditions, such as 33+/−1° C. A collection device such as a beaker or bowl is placed under hole 20. The dry mass of a second collection vessel is determined, and this vessel is located to catch any runoff that passes over the water impermeable material. Valve 24 is opened to dispense the 50 milliliters of distilled water contained in funnel 22 over a period of about 15 seconds. The mass of the collection vessel plus any liquid it collected is then determined. The mass of the liquid in the collection vessel is determined by subtracting the mass of the empty vessel from the mass of the full vessel after the test. This liquid mass is recorded as the runoff value. If liquid ran off the surface of the specimen but was not collected in the weighed collection vessel, the data point for that specimen should be discarded, and a new specimen tested in its place. Typically, the absorbent material is changed between insults, and the face of inclined surface 14 is dried. Each material specimen is tested only once. The test liquid is also discarded after one use. After a test, the film tape is removed from the lower edge of the used material specimen, and is set aside for the next specimen. Tape should be changed when it no longer adheres to specimens, or when the edges or surface of the tape are no longer smooth enough to allow runoff liquid to flow smoothly onto the impermeable material. To account for any material variability, at least three specimens per sample are tested, and the runoff amounts of the specimens are averaged.

As can be seen from reference to table 1, samples of article outer layers were tested according to the Hydrophilicity Test set forth above. The outer layer samples according to the present invention included a hydrophilic region that defined a runoff value of less than 10 grams, and in particular a runoff value of less than 5. As mentioned above, the runoff value is obtained by averaging the runoff of at least 3 specimens of a particular sample. Thus, as previously described, such a level of hydrophilicity (demonstrated by the runoff value) in the hydrophilic region of the outer layer will provide an article where the graphics disposed on the exterior article surface are more readily visible from the exterior article surface than if the outer layer did not include the hydrophilic region.

TABLE 1

| | | Runoff (g | Sample Average |
|---|---|---|---|
| Sample | Specimen | liquid) | Runoff (g liquid) |
| 0.5 osy (x gsm) polypropylene | | | |
| spunbound, untreated | | | |
| | 1 | 40.5 | |
| | 2 | 46.3 | |
| | 3 | 39.4 | |
| | | | 42.1 |
| 0.5 osy (17 gsm) polypropyl- | | | |
| ene spunbound, treated with | | | |
| Ahcovel Base N-62 and Glu- | | | |
| copan 220 surfactant mixture | | | |
| at an add-on of 0.38 weight | | | |
| percent. | | | |
| | 1 | 3.5 | |
| | 2 | 6.4 | |
| | 3 | 4.3 | |
| | | | 4.7 |
| The outer layer of Commer- | | | |
| cial swim pants: PAMP- | | | |
| ERS ® SUNNIES ® | | | |
| swim pants, available in the | | | |
| U.K. and obtained in Feb. of | | | |
| 2004. | | | |
| | 1 | 17.5 | |
| | 2 | 20.9 | |
| | 3 | 7.2 | |
| | | | 15.2 |
| | 4 | 7.8 | |
| | 5 | 22.4 | |
| | 6 | 16.1 | |
| | | | 15.4 |
| | 7 | 4.7 | |
| | 8 | 25.6 | |
| | 9 | 17.4 | |
| | | | 14.3 |

### Wet and Dry Bulk Thickness

Open the product and cut any elastics (e.g., leg elastics) so that the product can be laid flat. The product is sectioned into three parts, front, center and back, so that the absorbent pad is equally divided among the three sections. Using an AMES thickness gauge under 0.2 pounds per square inch (psi) pressure (14 grams per square cm), the thickness can be measured for each section, the front, center and back, and recorded. Each section should be spread flat to open any folds or wrinkles, but should not be stretched or pressed.

The sections are each then submerged in an ample excess of distilled water for ½ hour. Each section should be tested in a separate water bath, and the water should not be reused for subsequent tests. Where superabsorbent is present in an absorbent, or where the absorbent otherwise exhibits poor integrity when wet, the absorbent may need to be wrapped or enclosed in a sleeve or similar structure to prevent disintegration or movement of the absorbent in water or upon handling. The sleeve should be a non-swelling material with no surface active agents such as an untreated nonwoven web, and should be fitted closely to the absorbent section, but not so close as to inhibit any swelling of the absorbent. The sections are removed from the bath and placed horizontally on open screen for 5 minutes to allow excess water to drip out of each section. Each section is then measured under the AMES thickness gauge using 0.2 psi and recorded. If a sleeve is used, the bulk of the sleeve should be measured separately and subtracted from any bulk measurements that include both absorbent and sleeve.

As various changes could be made in the above constructions and methods, without departing from the scope of the invention, it is intended that all matter contained in the above description and shown in the accompanying drawings shall be interpreted as illustrative and not in a limiting sense.

When introducing elements of the invention or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed elements.

1. An absorbent article defining an interior article surface and an exterior article surface opposite the interior surface, said article comprising:
An absorbent body disposed toward said interior article surface;
An outer layer forming at least a portion of said exterior article surface;
A liquid impermeable layer located between said absorbent body and said outer layer; and
At least one graphic disposed on said exterior article surface wherein said outer layer defines a hydrophilic region that is coextensive with at least a portion of said at least one graphic.

2. The absorbent article of claim 1 wherein at least a portion of said liquid impermeable layer is attached to said outer layer.

3. The article of claim 1 wherein said graphic is applied on said liquid impermeable layer.

4. The article of claim 1 wherein said graphic is applied on said outer layer.

5. The article of claim 1 wherein said graphic is a multicolored graphic.

6. The article of claim 1 wherein said hydrophilic region comprises a nonwoven material.

7. The article of claim 1 wherein said hydrophilic region comprises natural fibers.

8. The article of claim 1 wherein said hydrophilic region comprises a surfactant.

9. The article of claim 1 wherein said hydrophilic region defines a hydrophilicity of less than 4 grams of runoff as determined by the Hydrophilicity Test set forth herein.

10. The article of claim 1 wherein said absorbent body comprises less than 25 weight percent super absorbent material.

11. The article of claim 1 wherein said absorbent body comprises between 0 and 10 weight percent super absorbent material.

12. The article of claim 1 wherein said absorbent body defines a wet bulk thickness of less than 1 cm as determined by the Bulk Thickness test set forth herein.

13. The article of claim 1 wherein said hydrophilic region comprises substantially all of said outer layer.

14. The article of claim 1 further comprising a bodyside liner disposed toward said interior article surface relative to said absorbent body wherein said bodyside liner is provided by a different material than said outer layer.

15. The article of claim 1 wherein said outer layer is provided by at least a portion of a garment shell, said garment shell defining a waist opening and a pair of hanging legs.

16. A disposable absorbent article defining an interior article surface and an exterior article surface opposite the interior surface, a first waist region, a second waist region, and a crotch region connecting the first and second waist regions, said article comprising:
An absorbent body disposed toward said interior article surface;
An outer layer forming at least a portion of said exterior article surface;
A liquid impermeable layer located between said absorbent body and said outer layer;
At least one graphic disposed on said exterior article surface wherein said outer layer defines a hydrophilic region that is coextensive with at least a portion of said at least one graphic; and
Side panels extending from and connecting said first and second waist regions in a pant configuration to form a waist opening and a pair of leg openings.

17. The absorbent article of claim 16 wherein at least a portion of said liquid impermeable layer is attached to said outer layer.

18. The article of claim 16 wherein said graphic is a multicolored graphic applied on said liquid impermeable layer.

19. The article of claim 16 wherein said hydrophilic region defines a hydrophilicity of less than 4 grams of runoff as determined by the Hydrophilicity Test set forth herein.

20. The article of claim 16 wherein said absorbent body comprises less than 30 weight percent super absorbent material.

21. The article of claim 16 wherein said absorbent body comprises between 0 and 10 weight percent super absorbent material.

22. The article of claim 16 wherein said absorbent body defines a wet bulk thickness of less than 1 cm as determined by the Bulk Thickness test set forth herein.

23. The article of claim 16 wherein said outer layer is provided by at least a portion of a garment shell, said garment shell defining a waist opening and a pair of hanging legs.
